(19) United States
(12) Patent Application Publication
(10) Pub. No.: US 2007/0161921 A1
(43) Pub. Date: Jul. 12, 2007

(54) Bio-accurate temperature measurement device and method of quantitatively normalizing a body temperature measurement to determine a physiologically significant temperature event

(76) Inventor: Jeffrey L. Rausch, Augusta, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/434,294

(22) Filed: May 15, 2006

Related U.S. Application Data
(60) Provisional application No. 60/756,864, filed on Jan. 7, 2006.

(51) Int. Cl. A61B 5/00; A61B 10/00

(52) U.S. Cl. 600/549; 600/300; 128/920; 600/551

(57) ABSTRACT

A device for determining normalized body temperature comprising a temperature sensor, an input device, a processor configured with a temperature-normalizing algorithm, memory, and an output device is described herein. Also disclosed is a method for determining physiologically significant changes in body temperature comprising providing raw body temperature of a subject, providing data for a temperature-normalizing algorithm, quantitatively normalizing the raw body temperature with an algorithm comprising an equation containing at least one body temperature-affecting variable to obtain a normalized body temperature, and comparing the normalized body temperature to a second temperature.

### CROSS-REFERENCE TO RELATED APPLICATIONS

This application claims the benefit of U.S. provisional application Ser. No. 60/756,864, filed Jan. 7, 2006, hereby incorporated by reference herein in its entirety for all of its teachings.

### BACKGROUND

Body temperature is a basic physiological measurement. There are many methods and devices for determining body temperature. These devices can be used in various locations in or on the body. Contact and non-contact temperature measuring devices are known and include, for example, the familiar glass and liquid thermometer, contact liquid crystal strips that change color, and electronic thermometers.

“Normal” body temperature in a human subject is generally thought of as 37° C. (98.6° F.); however, this temperature is actually a population average oral temperature. An individual's temperature varies naturally from factors other than disease or illness. Age, gender, activity level, time of year, and time of day are a few example variables that affect body temperature. There are also natural “normal” temperature variations between individuals. Natural temperature variations can introduce “noise” into a temperature reading, which is problematic when the reading is being used for purposes of identifying deviations or variations in temperature to diagnose disease or other physiological events or conditions.

The problem with determining what is a “normal” temperature stems from the fact that temperature, like all other physiologic and chemical measurements in humans, is expressed by a range of values, which can be normalized to the time of day (Mackowiak, P. A., Wasserman, S. S. (1995). Physicians' perceptions regarding body temperature in health and disease. South Med J 88: 934-938), age of the patient (Gomolin, I. H., Aung, M. M., Wolf-Klein, G., Auerbach, C. (2005). Older is colder: temperature range and variation in older people. J Am Geriatr Soc 53: 2170-2172; Smith, L. S. (2003). Reexamining age, race, site, and thermometer type as variables affecting temperature measurement in adults—A comparison study. BMC Nurs 2: 1; Takayama, J. I., Teng, W., Uyemoto, J., Newman, T. B., Pantell, R. H. (2000). Body temperature of newborns: what is normal? Clin Pediatr (Phila) 39: 503-510), gender (Baker, F. C., Waner, J. I., Vieira, E. F., Taylor, S. R., Driver, H. S., Mitchell, D. (2001). Sleep and 24 hour body temperatures: a comparison in young men, naturally cycling women and women taking hormonal contraceptives. J Physiol 530: 565-574), ovarian status (Coyne, M. D., Kesick, C. M., Doherty, T. J., Kolka, M. A., Stephenson, L. A. (2000). Circadian rhythm changes in core temperature over the menstrual cycle: method for noninvasive monitoring. Am J Physiol Regul Integr Comp Physiol 279: R1316-R1320), and expected interindividual variability (Letter (2005). My everyday body temperature is 97.4 degrees F., below the normal 98.6 degrees F. (37 degrees C.). So am I running a fever if my temperature is 99 degrees F? Johns Hopkins Med Lett Health After 50 17: 8; Sund-Levander, M., Grodzinsky, E., Loyd, D., Wahren, L. K. (2004). Errors in body temperature assessment related to individual variation, measuring technique and equipment. Int J Nurs Pract 10: 216-223). Ninety-eight point six degrees F. is not normal for all persons (Mackowiak, P. A., Wasserman, S. S., Levine, M. M. (1992). A critical appraisal of 98.6 degrees F., the upper limit of the normal body temperature, and other legacies of Carl Reinhold August Wunderlich. JAMA 268: 1578-1580), and 98.6° F. can even be a fever in certain contexts (Downton, J. H., Andrews, K., Puxty, J. A. (1987). ‘Silent’ pyrexia in the elderly. Age Ageing 16: 41-44; Higgins, P. (1983). Can 98.6 degrees be a fever in disguise? Geriatr Nurs 4: 101-102).

The difficulty in accounting for age effects on body temperature has led some authors to suggest a variety of different normal temperature values to be used for different ages (Castle, S. C., Norman, D. C., Yeh, M., Miller, D., Yoshikawa, T. T. (1991). Fever response in elderly nursing home residents: are the older truly colder? J Am Geriatr Soc 39: 853-857; Herzog, L. W., Coyne, L. J. (1993). What is fever? Normal temperature in infants less than 3 months old. Clin Pediatr (Phila) 32: 142-146). For example, older subjects have mean oral body temperatures lower than 98.6° F. Relatively few even achieve this temperature (Gomolin, et al., 2005). The literature now recommends abandonment of 98.6° F. as a relevant concept to clinical thermometry (Mackowiak, et al., 1992).

Temperature deviations are used as key signs of illness. Errors in body temperature assessment can seriously influence the evaluation of an individual's health condition (Sund-Levander, M., Grodzinsky, E., Loyd, D., Wahren, L. K. (2004). Errors in body temperature assessment related to individual variation, measuring technique and equipment. Int J Nurs Pract 10: 216-223). Certain febrile patients may not be reliably detected solely by a focused physical examination (Hung, O. L., Kwon, N. S., Cole, A. E., Dacpano, G. R., Wu, T., Chiang, W. K., et al. (2000). Evaluation of the physician's ability to recognize the presence or absence of anemia, fever, and jaundice. Acad Emerg Med 7: 146-156). Recent international vigilance regarding disease assessment has made attention to accurate measurement of body temperature increasingly important (Smith, L. S., 2003). However, even among physicians, no standardized or automated method exists to account for the many sources of temperature variation that may mask the identification of relevant body temperature markers.

Physicians differ substantially in their knowledge of, and attitude toward, body temperature and fever (Al Eissa, Y. A., Al Zaben, A. A., Al Wakeel, A. S., Al Alola, S. A., Al Shaalan, M. A., Al Amir, A. A., et al. (2001). Physician's perceptions of fever in children. Facts and myths. Saudi Med J 22: 124-128). Previous surveys indicate that a significant number of physicians show a serious lack of knowledge of the nature, dangers, and management of fever as an extremely common health problem (Al Eissa, et al., 2001). If asked to define fever, most physicians would offer a thermal definition, such as “fever is a temperature greater than . . . ” In offering their definition, many would ignore the significance of the age, gender, and diurnal oscillations that characterize body temperature variance (Mackowiak, P. A. (1998). Concepts of fever. Arch Intern Med 158: 1870-1881).

One survey of 268 physicians found that although 98% believed that body temperature normally varies during the day, there was no consensus of the magnitude of such variability (Mackowiak and Wasserman, 1995), let alone any method for normalizing the results within circadian context (Agarwal, S. K. (1980). Beware of the temperature chart. JAMA 243: 31-32). There was also considerable disagreement as to the specific temperatures defining the lower and upper limits of the febrile range (Mackowiak and Wasserman, 1995).

In another survey of 88 pediatric emergency registered nurses, the temperature considered to be fever ranged from 99.0° F. to 102.0° F., while the range considered dangerous ranged from 100.4° F. to 107.0° F. Eleven percent of these nurses were not sure what constituted a fever, and 31% were not sure what temperature would be dangerous (Poirier M P, Davis P H, Gonzalez-del Rey J A, Monroe K W (2000). Pediatric emergency department nurses' perspectives on fever in children. Pediatr Emerg Care 16: 9-12).

Confusion over what constitutes a normal body temperature also has an impact for society at large, beyond that related to health care. Surveys of caregivers show that 52% would unnecessarily check their child's temperature every hour or even more frequently when their child had a fever, 25% would give antipyretics for temperatures <100° F., and 85% would awaken their child to give antipyretics (Crocetti, M., Moghbeli, N., Serwint, J. (2001). Fever phobia revisited: have parental misconceptions about fever changed in 20 years? Pediatrics 107: 1241-1246). The consequences of parental fear included not only the unnecessarily frequent temperature measurements, but also sleeping in the same room (24%) and 13% remaining awake at night (van Stuijvenberg, M., de Vos, S., Tjiang, G. C., Steyerberg, E. W., Derksen-Lubsen, G., Moll, H. A. (1999). Parents' fear regarding fever and febrile seizures. Acta Paediatr 88: 618-622).

Temperature is also important for reasons other than the identification of fever. For example, temperature changes can indicate ovulation, metabolic disorders, and other conditions or events. It has also been recently found that depressed patients have an elevated temperature relative to non-depressed patients. See Rausch, J. L., Johnson, M. E., Corley, K. M., Hobby, H. M., Shendarkar, N., Fei, Y., Ganaphthy, V., Leibach, F. H., Depressed Patients Have Higher Body Temperature: 5-HT Transporter Long Promoter Region Effects Neuropsychobiology (2003) 47:120-127.

Even though temperature is known to be important and that small differences may be of interest, small variations are generally ignored because a clinician cannot readily determine what amount of a temperature variation is to be attributed to each potential cause.

Though it is known by those of skill in the art that various factors can affect body temperature (e.g., location on/in body where measurement is taken, gender, time of day, menstrual cycle, time of year/seasonal, activity level, eating, environment, medication, emotion, and age), these factors, at best, are sometimes informally and roughly taken into account. For example, a temperature of 103° F. in a geriatric patient may cause more alarm than a temperature of 103° F. in an infant patient. To date, the solution to this problem has been that clinicians are recommended to apply different suggested normative value ranges to different age patients and to qualitatively factor in time of day (with little or no guidance as to gender). However, this is virtually never done in practice, largely because it is a complicated process.

In the past, when 98.6° F. was thought to be normal, it was easy to simply assess whether a temperature measurement was significantly different from that value. However, now that identification of temperature-affecting factors has occurred, there is a need to develop a solution that takes these factors into account and reports temperature within its expected normative physiological context for a given individual's situation.

A way of more accurately diagnosing or predicting various physiologically important events based on temperature would be a very advantageous contribution to medicine. The current invention provides a system of measuring temperature and reporting measurements which takes into account or discounts factors influencing body temperature.

### SUMMARY OF THE INVENTION

Described herein is a device and a method for normalizing body temperature. The invention can include a device for determining a normalized body temperature of a subject comprising a temperature sensor for sensing raw body temperature of a subject, an input/output (I/O) interface configured to receive the sensed raw body temperature from the temperature sensor, and a processor configured to receive the sensed raw body temperature via the I/O interface and configured to perform a temperature normalizing algorithm to obtain a normalized body temperature. A device of the invention can further comprise one or more of a temperature sensor, an input device, a memory device, and an output device.

In one aspect, a device for determining a normalized body temperature of a subject comprises a temperature sensor for sensing raw body temperature of a subject, an input device for entering temperature-affecting variable information for calculation in a quantitative temperature-normalizing algorithm, a processor configured to perform the quantitative temperature-normalizing algorithm wherein the algorithm normalizes the raw body temperature to account for body temperature-affecting variables not of interest, a memory device, and an output device which provides the normalized body temperature in a usable format. The memory can store a variety of information, e.g, data and/or computer code.

In another aspect, a method for normalizing body temperature of a subject comprises providing a raw body temperature (T_{R}) of a subject, quantitatively normalizing the raw body temperature (T_{R}) with a temperature-normalizing algorithm wherein the algorithm comprises an equation containing at least one body temperature-affecting variable to obtain a normalized body temperature (T_{BA}).

In a further aspect, a method for determining physiologically significant changes in body temperature of a subject comprises providing a raw body temperature (T_{R}) of a subject, providing data for a temperature-normalizing algorithm, quantitatively normalizing the raw body temperature (T_{R}) with the algorithm wherein the algorithm comprises an equation containing at least one body temperature-affecting variable to obtain a normalized body temperature (T_{BA}), and comparing the normalized body temperature (T_{BA}) to a second temperature.

A method of the invention can be used to determine physiologically significant body temperature changes due to a physiologic condition or event such as fever, immune response, inflammatory disease, metabolic disorder, depression, or ovulation.

In yet another aspect the invention can include a computer program product for normalizing body temperature, the program being embodied on a computer-readable medium, on which is carried the program comprising a code segment comprising a quantitative temperature-normalizing algorithm.

A device and method of the invention allow for meaningful comparisons of normalized temperature, e.g., male person A to female person B or between a normalized temperature at time=0 (T₀) and an normalized temperature at time=t (Tₜ) for a single person A. These normalized temperature comparisons more readily show “real” temperature variations, i.e., indicating a physiological condition or event of interest.

Additional advantages will be set forth in part in the description which follows, and in part will be apparent from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 illustrates a block diagram of an example embodiment of a device of the invention.

FIG. 2 shows a flowchart representing an example embodiment of a method of the invention.

### DETAILED DESCRIPTION

Before the present compounds, compositions, articles, devices, and/or methods are disclosed and described, it is to be understood that the aspects described below are not limited to specific example embodiments disclosed. It is also to be understood that the terminology used herein is for the purpose of describing particular aspects only and is not intended to be limiting.

In this specification and in the claims which follow, reference will be made to a number of terms which shall be defined to have the following meanings:

It must be noted that, as used in the specification and the appended claims, the singular forms “a,” “an,” and “the” include plural referents unless the context clearly dictates otherwise. Thus, for example, reference to “an input device” includes more than one input device, reference to “a processor” includes more than one processor, and the like.

“Optional” or “optionally” means that the subsequently described event or circumstance may or may not occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used throughout, a “subject” means an individual. Thus, the “subject” can include a human. “Subject” can also include, for example, domesticated animals (e.g., cats, dogs, etc.); livestock (e.g., cattle, horses, pigs, poultry, sheep, goats, etc.); and laboratory animals (e.g., primate, mouse, rabbit, rat, guinea pig, etc.).

The term “raw body temperature” or “raw temperature,” as used herein, is intended to mean a subject's actual measured body temperature that has not been varied.

The term “normalized body temperature” or “normalized temperature,” as used herein, is intended to mean a body temperature value varied in accordance with the invention.

A current device and method of the invention allow for “apples to apples” comparisons of body temperature measurements, e.g., person to person or in a particular individual between time 0 and time t. Therefore, these normalized body temperature comparisons more readily show variations of interest as opposed to “noise” introduced by variables not of interest (e.g., febrile conditions as opposed to age of the subject).

Raw body temperature is the actual temperature of a subject, but what makes body temperature important for various applications are differences in one temperature relative to another temperature, e.g., measured raw temperature versus “normal” or average. The temperature normalization in the present invention can account for variations in temperature relative to various baseline temperatures. Example baseline temperatures are an individual's average or “normal” temperature, a subject's temperature at a particular time of day, or a population's average temperature.

A device or method of the present invention can provide real-time temperature information or information can be stored and evaluated at a user's convenience.

A. Device

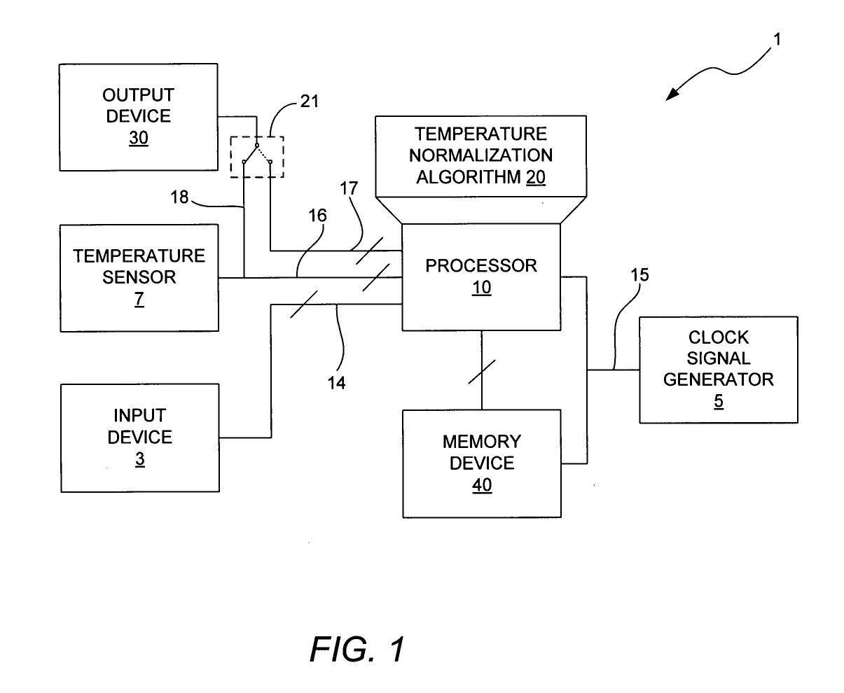

FIG. 1 illustrates a block diagram of an example embodiment of a temperature measuring and normalizing device 1 (aka “bio-accurate” temperature measurement device (BATM)) of the present invention.

The BATM device 1 can comprise a processor 10. The processor 10 performs a temperature-normalizing algorithm 20 (e.g., Equation 1), discussed further below. The processor 10 can be any type of computational device suitable for performing the temperature-normalizing algorithm 20. For example, the processor 10 can be a microprocessor, a microcontroller, an application specific integrated circuit (ASIC), a field programmable gate array, a programmable logic array, and/or a combination of discrete components. The processor 10 can be hardware, software, or a combination of hardware and software or firmware. The processor 10 typically is a microprocessor that executes a software computer program that performs the algorithm 20. The processor 10 can receive and process a signal from a temperature sensor 7 to determine raw body temperature. The processor 10 can send a signal to an output device 30. Processors are commercially available, and one of skill in the art can determine an appropriate processor for a particular embodiment of the device.

The processor 10 can receive a raw body temperature signal via a line 16 from a temperature sensor 7. The temperature sensor 7 can include various conventional sensors or those yet to be developed, e.g., contact or no contact sensor, thermocouple, infrared, oral, rectal, tympanic, axillary, ingestible or implantable core body temperature pill, and/or combination thereof. Temperature sensors are commercially available, and one of skill in the art can determine an appropriate temperature sensor for a particular embodiment of the device.

The BATM device 1 can, and typically will, comprise an input device 3, e.g., keypad, keyboard, clock, port, and/or combination thereof. An input device 3 can be used to input temperature-affecting information or data (such as gender, age, and time of day, as illustrated below in the example temperature normalization equation, Eqn. 1) via one or more lines 14, which can be connected to the processor 10 via an input/output (I/O) interface of device 1. For example, a keypad can be used to input temperature-affecting information gender (G) and age (A) and a clock used to input time (t) in a particular embodiment of a device of the invention (FIG. 1). Input devices are commercially available, and one of skill in the art can determine appropriate input devices for a particular embodiment of the device.

In an example embodiment, the BATM device 1 includes a clock signal generator 5. The processor 10 typically receives a clock signal via a line 15 from a clock signal generator 5, which the processor 10 can use to calculate, for example, time of day. In an example embodiment, the algorithm 20 includes functionality for associating time of day with each raw temperature value and/or each normalized temperature value.

The processor 10 can, for example, receive a raw body temperature signal, a clock signal, and temperature-affecting information from an input device 3 and perform the algorithm 20 to produce a normalized temperature value. A signal from the temperature sensor 7 can be converted to a raw body temperature. One of skill in the art can determine an equation or algorithm for converting the sensor signal to a raw body temperature. This body temperature as measured by the sensor is also referred to herein as the “raw temperature.”

An algorithm 20 can be used to quantitatively normalize the raw temperature to a normalized temperature (aka “bio-accurate” temperature). An algorithm 20 for quantitatively normalizing temperature can include an equation which has a term T_{R} which is the measured body temperature (raw temperature) from a sensor 7 and also has terms for at least one variable which affects body temperature. Variables that can affect body temperature include, but are not limited to, age, gender, and time of day. The following temperature normalization equation (Equation 1) can be used in an example embodiment of the invention to determine a normalized body temperature:
T_{BA}=T_{R}−0.104G+0.0107A+0.549(sin(2πt))−0.614(cos(2πt))−1.172 (Eqn. 1)
wherein
T_{BA}=“bio-accurate” (normalized) temperature,
T_{R}=raw temperature,
G=gender (1=male, 2=female),
A=age (years), and
t=time of day (in decimal proportion of the day). In this example, the equation corrects to a normal temperature being reported as the traditional value of 98.6° F. The algorithm 20 (or, in an example embodiment, the T_{BA} equation) can be updated, for example, as more data and more studies show additional factors influencing temperature or provide refinement of the coefficients and refinement of the mathematical method for temperature normalization (e.g., inclusion of second-order harmonics into the circadian factor). The variable values other than raw temperature can be provided by an input device 3 and these values can be from a measurement device, input by a user, or from a database, for example. The algorithm 20 of the device 1 can further include an equation for calculating temperature difference (Equation 2), e.g.,
ΔT_{BA}=T_{BA2}−T_{BA1}. (Eqn. 2)
An equation for normalized temperature difference can calculate the difference between a normalized temperature reading (T_{BA2}) and a temperature “baseline” (T_{BA1}) or a second normalized temperature reading (T_{BA1}) to determine changes in temperature. For example, a temperature baseline can be the traditional 98.6° F. (37° C.) body temperature population average or an average temperature for a specific individual. Also, T_{BA1} can be, for example, a normalized temperature reading at an earlier point in time or for a different individual.

An algorithm 20 of the invention can further include other equations. In an example embodiment, the algorithm can select the “best” temperature normalization (i.e., a population-based temperature normalization (e.g., Equation 1) or a subject-based temperature normalization) for a given temperature measurement for a particular subject. The selection of best temperature normalization is dependent upon how much data has been accrued for that particular subject. In an example embodiment, a device of the invention can determine, store, and analyze repeated measures of temperature in a known particular subject.

A particular subject can be identified, for example, by typing identifying information, such as a number, in response to a prompt from the device 1. Once prompted, a user can, e.g., press an “ID” button on an input device 3 and enter identifying information for that individual, e.g., 1=individual A, 2=individual B, and the like.

An advantage of the ability to determine, store, and analyze repeated measures of temperature in a known subject is that the device “learns” the expected temperatures for that individual subject. As the number of temperature measurements increases, it becomes increasingly probable that a temperature normalization based on the accrued temperature values for that subject will more accurately identify a clinically significant departure from normal temperature for that subject than would a temperature normalization from a population-based normalization equation. Thus, a population-based temperature normalization will typically best serve cases where single temperatures are being taken in a variety of subjects, and an individual-based normalization will typically best serve cases where temperature is being taken multiple times in a single individual.

In an example embodiment, the device can determine whether a more accurate temperature correction would result from using a time-of-day normalization developed from population-based data or from using a time-of-day normalization derived from multiple measures in an individual (assuming a statistically sufficient number of different times of day and corresponding temperatures have been recorded for that individual). In cases where there are not enough different times of day with corresponding temperatures that have been recorded for an individual, the device can use a population-based time normalization and still determine whether or not to use the other population-based normalization variables (e.g., age and gender).

During a course of measuring and recording body temperature for a given individual over time, there can be a progressive sequence of appropriate temperature normalization equations (e.g., population-based normalization followed by individual-based normalization as the number of data points increases for an individual and single variable (i.e., time-based) temperature normalization followed by multi-variable temperature normalization). It first becomes more probable that population-based time-normalized (single variable) temperatures will more accurately identify clinically significant temperature variance for an individual than would a population-based multi-variable (e.g., time, age and gender) temperature normalization. The progression from population-based single variable normalization to population-based multi-variable normalization is likely to happen sooner than a progression to using the individual's time of day (single variable) temperature normalization from a population-based time of day normalization. This is true because it will require a greater number of temperature measurements taken at different times of day to characterize an individual's circadian pattern than the number of measurements required to characterize an individual's own normal temperature for his/her given age and gender.

In other words, the more times a population-based time-normalized temperature is taken in the same subject, the more likely it is that a temperature deviation would be accurately identified as a real deviation for that person than a temperature normalized by a multi-variable population-based normalization.

The device can determine when repeated measures of population-based time-normalized temperature from an individual will give a better normalization than also normalizing for variables such as age and gender (given that, e.g., the gender and birth date of the individual are constants).

To do this, the subject's mean population-based time-normalized temperature (X) is compared each time to the subject's mean population-based multi-variable (time, gender, and age) normalized temperature (P). Each time (N=the number of data points) a temperature is measured, the current population-based time-normalized temperature (T) is compared first to X and then to P as follows:
If

$$\frac{\sum {( X - T )}^{2}}{N - 1} \qquad ( Eqn . 3 )$$

is less than

$$\frac{\sum {( P - T )}^{2}}{N} , \qquad ( Eqn . 4 )$$

then there is less variance from the population-based time normalization equation than the population-based multi-variable (time, age, and gender) normalization equation. Therefore, use of the population-based time-normalized temperature for that subject would more accurately identify a clinically significant temperature variance than would use of a population-based temperature normalization based on inclusion of the additional variables, such as age and gender. (Note the difference in the denominators; this is because the X term includes the current time point in each case.)

Alternatively,
if

$$\frac{\sum {( X - T )}^{2}}{N - 1} \qquad ( Eqn . 3 )$$

is greater than

$$\frac{\sum {( P - T )}^{2}}{N} , \qquad ( Eqn . 4 )$$

then the population-based multi-variable temperature normalization would be more accurate than would a population-based time-only normalization.

In an example embodiment, the device can also determine when an individual's own circadian pattern of temperature is the more accurate method of temperature normalization than use of the reference population's circadian pattern of temperature. To do this, the grand mean of the observed subject's individual time-normalized temperatures (Y) is first calculated by deriving the person's own individual constants for time variation using, for example, a standard multiple regression fit for cosinor analysis:
Tᵣ=B₀+B₁ cos(2πt)+B₂ sin(2πt), (Eqn. 5)
where Tᵣ=raw un-normalized temperature and B=constant derived from a regression fit.

The individual's own circadian pattern of temperature normalization for the current temperature (T_{ic}) is then calculated with the equation:
T_{ic}=Tᵣ−B₁ cos(2πt)−B₂ sin(2πt)−(Y−98.6), (Eqn. 6)
where Y=the grand mean of the measures of the individual's own circadian pattern of time-normalized temperature (T_{ic}) in Fahrenheit degrees.

The mean individual's own circadian time-normalized temperature (Y) is compared each time to the population's time-normalized temperature (X). Each time temperature is taken, the current population time-normalized temperature (T) is compared to first to Y and then to X as follows:
If

$$\frac{\sum {( Y - T )}^{2}}{N - 1} \qquad ( Eqn . 7 )$$

is less than

$$\frac{\sum {( X - T )}^{2}}{N} , \qquad ( Eqn . 3 )$$

then the individual's own circadian time-normalized temperatures for that subject would more accurately identify a clinically significant temperature variance than would a population-based time normalization. (Note the difference in the denominators which is because the Y term includes the current time point in each case.)

Conversely,
if

$$\frac{\sum {( Y - T )}^{2}}{N - 1} \qquad ( Eqn . 7 )$$

is greater than

$$\frac{\sum {( X - T )}^{2}}{N} , \qquad ( Eqn . 3 )$$

then the population-based time normalization would more accurately identify clinically significant temperature variance than would the individual's own circadian time-normalized temperature.

In sum, with respect to whether to use the multi-variable population-based correction, only the population-based circadian correction, or only the individual-based circadian correction, the more accurate method would be identified by which of the terms

$$\begin{matrix}\frac{\sum {( Y - T )}^{2}}{N - 1} & ( Eqn . 7 ) \\ \frac{\sum {( P - T )}^{2}}{N} , & ( Eqn . 3 ) \\ \frac{\sum {( X - T )}^{2}}{N - 1} & ( Eqn . 3 )\end{matrix}$$

yields the lowest number.

Consequently, a device of the current invention can “know” the “best” correction for a particular subject based on how much data has accrued for that subject.

A normalized temperature (T_{BA}) or normalized temperature difference (ΔT_{BA}) determined by the device 1 can be output, such as to an output device 30 and/or stored in a memory device 40 for future use by the processor 10 or future output.

An algorithm 20, or specifically an equation, for normalizing body temperature can be determined by, for example, providing temperature data from a population of subjects with various ages, genders, at various times, etc.; performing linear regression on the provided temperature, age, gender, time, etc. data to generate a relation between the variables (age, gender, time, etc.) and body temperature and to identify those variables with a significant effect on body temperature; and generating a temperature-normalizing equation with relevant temperature-affecting variables.

A BATM device 1 of the invention can, and typically does, include an output device 30, e.g., screen, USB/serial/parallel port, audio device, and/or combinations thereof. An output device 30, e.g., a light emitting diode (LED) display device, can, in an example embodiment, visually display the normalized temperature calculated by the processor 10 and output via a line 17 to the output device 30. An indication of the raw temperature value can be output via a line 18 to the output device 30. In an example embodiment, raw temperature and normalized temperature can be displayed simultaneously on the output device 30. Alternatively, the BATM device 1 can, e.g., include a switch 21 to enable a user to select the output on line 17 or the output on line 18 to be provided to the output device 30. A switch 21 can be labeled to allow a user to easily discern whether raw or normalized temperature is being displayed. A visual output device 30 can be configured such that various temperature readings appear in different colors as to be readily understandable to a user—e.g., yellow reading=raw temperature, blue reading=low or decreasing temperature, red reading=high or increasing temperature. In another example, an audio output device 30 can be configured to sound an “alarm” in response to an abnormal adjusted temperature (e.g., parental monitoring of fever in an infant). Output devices are commercially available, and one of skill in the art can determine appropriate output devices for a particular embodiment of the device.

A BATM device 1 of the invention can, and typically does, include a memory device 40. The memory device 40 can, for example, store instructions of a software program that performs algorithm 20 and data. The memory device 40 can be any type of memory device, e.g., random access memory (RAM), dynamic RAM (DRAM), flash memory, read only memory (ROM), compact disk ROM (CD-ROM), digital video disk (DVD), magnetic disk, magnetic tape, and/or a combination thereof. A device of the invention also encompasses electrical signals modulated on wired and wireless carriers (e.g., electrical conductors, wireless carrier waves, etc.) in packets and in non-packet formats. Memory could be used, for example, to data log temperatures for a particular individual over time. Memory is commercially available, and one of skill in the art can determine an appropriate type and amount of memory for a particular embodiment of the device.

A device 1 of the invention can further include a power source, e.g., battery. Power sources are commercially available, and one of skill in the art can determine appropriate power sources for a particular embodiment of the device.

A device 1 of the invention can comprise optional additional components. Optional components are commercially available, and one of skill in the art can determine appropriate optional components for a particular embodiment of the device.

Appropriate electrical/communication and/or mechanical connections between the components of the device can be chosen by one of ordinary skill in the art for a particular embodiment of the invention.

Signals can be transmitted between components of the device and/or external devices/components using conventional devices or means.

A device of the invention can be constructed according to procedures known to one of ordinary skill in the art.

In an embodiment of a device of the invention, the device can be connected to a personal computer or personal digital assistant, for example. A computer could be used to store information from the device or download information to the device (e.g., an updated or new algorithm 20), for example.

The normalized temperature determined by the algorithm 20 (or other information output from a device 1 of the invention) can be used in a conventional clinical decision-making process to determine the probability of the temperature of a subject being related to a disease, condition, or event of interest rather than those “normal” variations in temperature.

In another example embodiment, the invention can include a computer program product for normalizing body temperature, the program being embodied on a computer-readable medium, on which is carried the program comprising a code segment comprising a quantitative temperature-normalizing algorithm.

B. Method

Since it is known in the art that body temperature varies for reasons other than, for example, illness or ovulation status, a need exists for a method of more easily distinguishing the variations (and extent of these variations) that indicate physical events of interest from those temperature variations which are simply “normal” deviations from averages.

A method of the current invention includes normalizing measured raw body temperature with factors accounting for, e.g., gender, age, and time of day. Gender, age, and circadian rhythms can add “noise” to a temperature measurement making it harder to recognize rising or falling temperatures, especially when these temperature deviations of interest may be only a degree or two from “normal.” For example, the change in basal body temperature indicating ovulation may only be 1° F. or less.

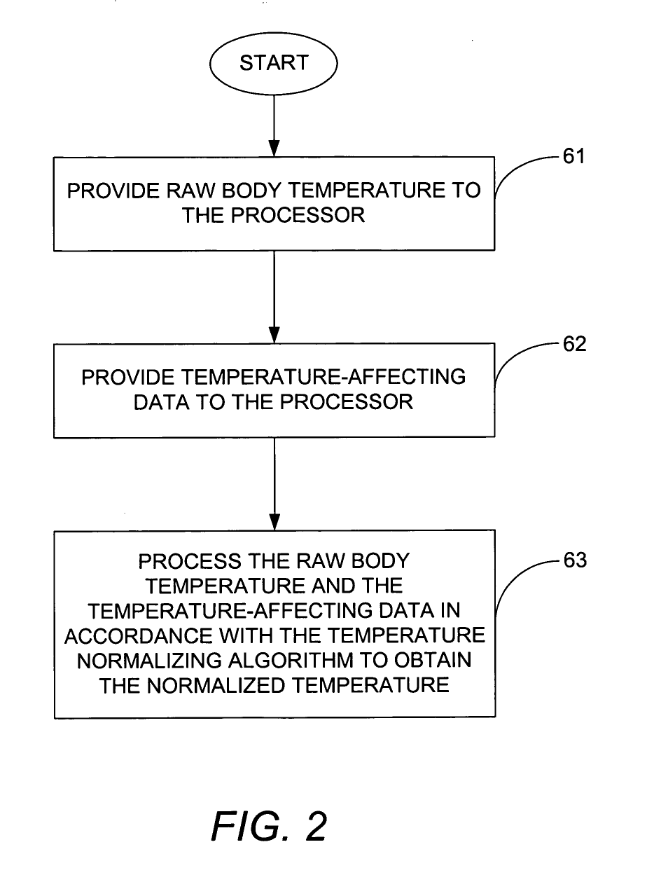

FIG. 2 illustrates a flowchart representing an example embodiment of a method of the invention. A raw body temperature of a subject is provided to a processor, as indicated by block 61. Variable data for the temperature-normalizing algorithm (e.g., gender, age, time) to be executed by the processor is provided to the processor, as indicated by block 62. The temperature-normalizing algorithm is then performed by the processor to process the raw temperature and variable data to obtain a normalized temperature, as indicated by block 63.

A method of the invention comprises providing a raw body temperature of a subject. Provision of the body temperature can be, for example, by measuring the body temperature of a subject. Various methods of providing a raw body temperature are known to one of ordinary skill in the art.

To identify meaningful small differences in temperature, a strict measurement protocol may be desirable when measuring a raw body temperature. Therefore, a method of the invention can further comprise conventional steps of strict temperature measurement protocol, e.g., no eating, no drinking, or activity by the subject for a period of time prior to measurement.

A method of the invention comprises providing data for variables in an algorithm for quantitatively normalizing temperature. Provision of the data for the variables (discussed above with the algorithm) can be by, for example, a user providing information or providing values from a database or a device.

A method of the invention comprises normalizing the raw body temperature using an algorithm. The raw body temperature can be quantitatively normalized (aka “bio-accurate” temperature). An algorithm for quantitatively normalizing temperature can include an equation which has a term T_{R} which is the raw body temperature and also has a term for at least one variable which affects body temperature. Variables that can affect body temperature include, but are not limited to, age, gender, and time of day. A temperature normalization equation (discussed above in Device section) can be used in an example embodiment of the method to determine a “bio-accurate” body temperature (normalized for various parameters which affect body temperature). The algorithm or T_{BA} equation can be updated, for example, as more data and more studies show additional factors influencing temperature or provide refinement of the coefficients and refinement of the mathematical method for temperature normalization (e.g., inclusion of second-order harmonics into the circadian factor). The variable values are provided (discussed above), and these values can be from a measurement device, input by a user, or from a database, for example. The algorithm of the method can further include an equation for temperature difference, e.g., equation 2 (discussed above in Device section). An equation for a normalized temperature difference can calculate the difference between a normalized temperature reading (T_{BA2}) and a temperature “baseline” (T_{BA1}) or a second normalized temperature reading (T_{BA1}) to determine changes in temperature. For example, a temperature baseline might be the traditional 98.6° F. (37° C.) body temperature population average or an average temperature for a specific individual. Also, e.g., T_{BA1} might be a normalized temperature reading at an earlier time or for a different individual.

An algorithm of the invention can also comprise determining the “best” temperature normalization for a given temperature measurement for a particular subject (discussed above in Device section, e.g., paras. [0042]-[0057]).

A method of the invention comprises providing a raw body temperature of a subject, providing data for variables in an algorithm for quantitatively normalizing temperature, and normalizing the raw body temperature using an algorithm. A method of the invention can further comprise determining an algorithm for normalizing body temperature. The determination of an algorithm can comprise, for example, providing temperature data from a population of subjects with various ages, genders, at various times, etc.; performing linear regression on the provided temperature, age, gender, time, etc. data to generate a relation between the variables (age, gender, time, etc.) and body temperature and to identify those variables with a significant effect on body temperature; and generating a temperature-normalizing equation with relevant temperature-affecting variables.

A method of the invention can further comprise comparing the normalized body temperature to a second body temperature to determine a body temperature difference. The second temperature can be a second normalized body temperature, a non-normalized body temperature, or a conventional (literature) body temperature (e.g., 37° C./98.6° F.).

A method of the invention can further comprise diagnosing or determining a physiological condition or event based on the normalized body temperature or normalized body temperature difference. Example physiological conditions or events correlating with body temperature are fever, ovulation, entry into menopause, depression, inflammatory disease, or metabolic disease. Temperature information can be combined with conventional techniques for diagnosing or identifying these physiological conditions or events, e.g., laboratory testing, imaging techniques, and/or patient history.

A correlation of temperature ranges or temperature differentials to a physiological condition or event can be determined by one of skill in the art or found in literature, e.g., an increased temperature correlates to fever or ovulation or decreased temperature correlates to hypothyroidism.

The invention can include a method of using normalized body temperature to predict ovulation. The invention can include a method of predicting ovulation comprising substituting normalized body temperature for raw body temperature in a conventional basal body temperature ovulation prediction method. Conventional basal body temperature ovulation prediction methods are known to one of ordinary skill in the art.

The invention can include a method of determining ovulation comprising determining normalized basal body temperature of a female subject in the morning prior to activity using a device of the invention, charting normalized basal body temperature over a period of time, and identifying a rise in normalized basal body temperature which correlates with ovulation having occurred. The invention can include a method of predicting ovulation comprising determining normalized basal body temperature of a female subject in the morning prior to activity using a device of the invention, charting normalized basal body temperature over a period of time, and identifying a decrease in normalized basal body temperature which precedes a predicted rise in normalized basal body temperature indicative of ovulation.

The invention can include a method of determining ovulation comprising determining raw basal body temperature of a female subject in the morning prior to activity, normalizing the raw basal body temperature using a quantitative temperature-normalizing algorithm, charting the normalized basal body temperature over a period of time, and identifying a rise in normalized basal body temperature which correlates with ovulation having occurred. The invention can include a method of predicting ovulation comprising determining raw basal body temperature of a female subject in the morning prior to activity, normalizing the raw basal body temperature using a quantitative temperature-normalizing algorithm, charting normalized basal body temperature over a period of time, and identifying a decrease in normalized basal body temperature which precedes a predicted rise in normalized basal body temperature indicative of ovulation.

An algorithm for quantitatively normalizing body temperature is described above.

The invention can include a method for diagnosing depression. An example method comprises taking or referring to a patient history including questions related to depression; performing a physical exam including at least taking the patient's temperature; normalizing the patient's temperature measurement using a quantitative temperature-normalizing algorithm of the invention; analyzing the history, physical exam, and normalized temperature information to determine the probability of depression based on known correlations of that information and depression.

C. Applications

Body temperature or a change in body temperature can indicate a variety of physiological events or conditions.

A method of the invention can be used to identify a body temperature (or body temperature change) correlating with depression. An increase in body temperature normalized for circadian rhythm, age, and gender has been found to correlate well with the incidence of depression. The normalized temperatures reveal increased body temperature in patients suffering from clinical depression. Use of body temperature, therefore, can be of assistance in diagnosing depression. See Rausch, J. L., Johnson, M. E., Corley, K. M., Hobby, H. M., Shendarkar, N., Fei, Y., Ganaphthy, V., Leibach, F. H., Depressed Patients have Higher Body Temperature: 5-HT Transporter Long Promoter Region Effects, Neuropsychobiology (2003) 47: 120-127 (demonstrated a 0.4° F. raw body temperature elevation—small compared to fever but identifiable over normal temperature with the use of precise controlled methods).

A method of the invention can be used to identify a body temperature (or body temperature change) correlating with ovulation, menses, or other hormonal events.

In the case of physical conditions or events only found in one gender, a gender factor of the algorithm can either be eliminated (since temperature differences (ΔT) are the same whether the factor is included or not) or can be set as the constant as calculated from the equation (e.g., 0.208 for women). A device or method for prediction of ovulation, for example, can use a simplified equation, e.g.,
T_{BA}=T_{R}−0.208+0.0107A+0.549(sin(2πt))−0.614(cos(2πt))−1.172. (Eqn. 8a)
T_{BA}=T_{R}+0.0107A+0.549(sin(2πt))−0.614(cos(2πt))−1.380. (Eqn. 8b)

A method of the invention can be used to identify a body temperature (or body temperature change) correlating with presence of fever or an immune response. A benefit of such a method could be early detection or detection where the illness or disease might otherwise be missed. An example method comprises taking or referring to a patient history including questions related to symptoms of fever or an immune response; performing a physical exam including at least taking the patient's temperature; normalizing the patient's temperature measurement using a quantitative temperature-normalizing algorithm of the invention; analyzing the history, physical exam, and normalized temperature information to determine the probability of fever or an immune response based on known correlations of that information and fever or an immune response.

A method of the invention can be used to identify a body temperature (or body temperature change) correlating with presence of inflammatory disease, e.g., chronic fatigue syndrome, fibromyalgia, or arthritis. An example method comprises taking or referring to a patient history including questions related to symptoms of inflammatory disease; performing a physical exam including at least taking the patient's temperature; normalizing the patient's temperature measurement using a quantitative temperature-normalizing algorithm of the invention; analyzing the history, physical exam, and normalized temperature information to determine the probability of inflammatory disease based on known correlations of that information and inflammatory disease.

A method of the invention can be used to identify a body temperature (or body temperature change) correlating with presence of other diseases with symptomatic changes in body temperature, e.g., hypothyroidism. An example method comprises taking or referring to a patient history including questions related to hypothyroidism; performing a physical exam including at least taking the patient's temperature; normalizing the patient's temperature measurement using a quantitative temperature-normalizing algorithm of the invention; analyzing the history, physical exam, and normalized temperature information to determine the probability of hypothyroidism based on known correlations of that information and hypothyroidism.

### EXAMPLES

The following examples are put forth so as to provide those of ordinary skill in the art with a complete disclosure and description of how the articles, devices, and/or methods described and claimed herein are used and are intended to be purely exemplary and are not intended to limit the scope of what the inventor regards as his invention. Efforts have been made to ensure accuracy with respect to numbers (e.g., amounts, temperature, etc.) but some errors and deviations should be accounted for. Unless indicated otherwise, parts are parts by weight, temperature is in ° F. or is at ambient temperature, and pressure is at or near atmospheric. There are numerous variations and combinations of conditions that can be used to optimize the described process. Only reasonable and routine experimentation will be required to optimize the processes.

### Example 1

### Prophetic Example Fever

An 89-year-old male is seen on medical rounds with a raw body temperature at 8:50 AM of 98.6° F., which is considered normal by his health care team. The next day his temperature is measured again at 8:45 AM, and this time his raw temperature is 99.9° F. The health care team refers to the literature which states that although anything above 98.6° F. can be considered a fever, it is not considered to be a medically significant fever unless it is 100.4° F. or higher, which it is not. Consequently, they consider that no action is necessary based on his body temperature, but order additional diagnostic tests to be sure. Over the next three days, the patient worsens with progressive weakness and malaise that make it difficult for him to fight a belatedly diagnosed infection for which antibiotics are given too late, and he eventually succumbs to his febrile illness.

The same 89-year-old male is seen on medical rounds with a raw body temperature at 8:50 AM of 98.6° F. However, his normalized “bio-accurate” body temperature is 99.1° F. His health care team recognizes 99.1° F. as a sign of potential fever that may not be medically significant, but orders additional diagnostic tests to be sure. The next day his temperature is measured again at 8:45 AM, and this time his raw body temperature is 99.9° F. His normalized “bio-accurate” body temperature is 100.4° F. His health care team recognizes 100.4° F. as a medically significant fever. They look at the results of yesterday's tests and order antibiotics to begin. The infection is caught in time. The infection is treated early enough, and the patient's strength is still sufficient to mount a good response to treatment.

### Example 2

### Prophetic Example Ovulation

A 26-year-old married female takes her temperature to determine if she is fertile. Her raw temperature taken at 9:36 PM is 99.4° F. She is aware that ovulation can cause a rise in body temperature of 0.45-0.81° F. and recognizing that her's is 0.8 degrees above normal, she changes her plans and makes extenuated efforts to conceive, but is disappointed in the lack of results.

The same 26-year-old married female takes her temperature to determine if she is fertile. Her raw temperature taken at 9:36 PM is 99.4° F. Her normalized “bio-accurate” temperature is 97.5°, indicating that she is not likely fertile. She maintains her plans for that day, better allowing for a subsequent free time to conceive later that month.

The same 26-year-old married female later takes her temperature again to determine if she is fertile. Her raw temperature taken at 8:49 AM is 99.4° F. Without the “bio-accurate” thermometer, her prior experience above may lead her to believe that she is not fertile, since this was the same raw temperature value obtained previously when she was not fertile. However, her normalized “bio-accurate” temperature for that time of day is 99.1° F. The “bio-accurate” thermometer reports to her that she may be fertile. She changes her plans and makes efforts to conceive, and now has the new baby for which she had hoped.

### Example 3

### Prophetic Example Hypothyroidism

A 30-year-old female complaining of fatigue has her temperature taken in the doctor's office at 4:48 PM with a raw uncorrected reading of 98.6° F., suggesting a normal reading. She indicates that she has been under a lot of stress and that the stress may explain her fatigue. However, her “bio-accurate” thermometer reading is 97.2° F., indicating a reading 1.40 below normal. Her doctor recognizes that the low temperature could be a sign of chronic fatigue syndrome or hypothyroidism and decides to order thyroid tests which indicate that she has hypothyroidism.

### Example 4

### Prophetic Example Depression

A 58-year-old male is seen for his yearly check-up at 8:50 AM. He denies any complaints except for trouble sleeping, but he says that is his own fault. He seems stressed and irritable, but the doctor attributes that to her running late that morning. His raw uncorrected temperature is 98.9° F. which seems unremarkable, within the day's variation. However, his normalized “bio-accurate” temperature is 99.1°, high enough to generate a suspicion of clinical depression. Although the doctor is running late, she decides to screen further for depression with additional questions beyond those usually asked during yearly check-up exams. She finds that he has had a persistently irritable mood for more than two weeks, insomnia, a reduction in interest in his usual activities, an exaggerated sense of guilt, often feels tired, and answers yes that he does note prominent intermittent difficulty in concentrating. She diagnoses clinical depression and starts antidepressant treatment, since the normalized temperature reading suggested that she screen more thoroughly for the presence of depression.

### Example 5

### Prophetic Example Peri-Menopause

A 38-year-old female is seen for her yearly check-up at 1:40 PM with a raw uncorrected body temperature of 98.6° F., and she denies any symptoms when first asked. However, her normalized “bio-accurate” body temperature is 97.9° F., which is low. This sparks further inquiry by her doctor, who finds no symptoms of hypothyroidism or fatigue. But, the patient does note hot flashes and irregular, infrequent periods when asked specifically about them. Although the doctor may have considered 38 years old to be likely too young for menopausal symptoms, he knows that menopausal women with hot flashes have lower body temperatures than similar such asymptomatic women. Because the temperature reading alerted him to a possible abnormality, he diagnoses peri-menopausal hot flashes and treats them successfully with sertraline.

### Example 6

### Pneumonia—Saving Medical Costs and Unnecessary Time and Risk with “Bio-Accurate” Temperature

A 23-year-old, obese female patient was seen in the clinic for depression. She had a raw body temperature of 100.3° F. and a cough. Physical exam of the lungs by the clinician found rales and rhonchi which was potentially consistent with her smoking status. However, she also had dullness to percussion in her right lower lung field.

Although the percussion dullness was consistent with obesity raising the height of her diaphragm or with liver enlargement from her past substance abuse, it was also potentially indicative of lung consolidation as a symptom of walking pneumonia. Since her raw temperature was very close to 100.4° F. (a clear traditional fever) and since raw temperature is subject to extraneous variation (as discussed above), a chest X-ray was ordered to rule out the presence of pneumonia.

Upon return to the clinic, her raw temperature at the same time of day (2:24 pm) was again 100.3° F., but the X-rays were negative with no radiographic suggestion of pneumonia. This time a normalized body temperature was calculated per a method of the invention using Equation 1. The raw temperature of 100.3° F. normalized to 99.3° F.

Therefore, this example demonstrates that if a method of normalizing body temperature of the invention had been used in the first instance, a potentially unnecessary medical test could have been avoided (the chest X-ray). This example illustrates potential value to a patient, health care provider, insurance company, managed care company, government, or other utilization management stakeholder of using normalized body temperature rather than simply measuring raw body temperature. This example, thus, illustrates potential for eliminating or reducing costs, exposure to radiation, and time for those involved in this example situation.

Throughout this application, various publications are referenced. The disclosures of these publications in their entireties are hereby incorporated by reference into this application in order to more fully describe the compounds, compositions and methods described herein.

Various modifications and variations can be made to the devices and methods described herein. Other aspects of the devices and methods described herein will be apparent from consideration of the specification and practice of the devices and methods disclosed herein. It is intended that the specification and examples be considered as exemplary.

What is claimed is:

1. A device for determining a normalized body temperature of a subject comprising
a) a temperature sensor for sensing raw body temperature of a subject,
b) an input/output (I/O) interface configured to receive the sensed raw body temperature from the temperature sensor, and
c) a processor configured to receive the sensed raw body temperature via the I/O interface and configured to perform a temperature-normalizing algorithm to obtain a normalized body temperature.

2. The device of claim 1 further comprising an input device, in communication with the I/O interface, for entering temperature-affecting variable information into the device for use by the processor in performing the temperature-normalizing algorithm.

3. The device of claim 1 further comprising an output device, in communication with the I/O interface, for receiving the normalized body temperature and providing the normalized body temperature in a manner accessible to a user.

4. The device of claim 1 further comprising a memory device.

5. The device of claim 2 wherein the input device is a keypad, a keyboard, clock, a port, or a combination thereof.

6. The device of claim 3 wherein the output device is a screen, a port, an audio device, or a combination thereof.

7. The device of claim 6 wherein the output device is a screen and the screen displays raw body temperature and/or normalized body temperature.

8. The device of claim 1 wherein the temperature-normalizing algorithm comprises a quantitative temperature-normalizing equation:
T_{BA}=T_{R}−0.104G+0.0107A+0.549(sin(2πt))−0.614(cos(2πt))−1.172,
wherein
T_{BA}=normalized body temperature,
T_{R}=raw body temperature,
G=gender (1=male, 2=female),
A=age (years), and
t=time of day (in decimal proportion of the day).

9. The device of claim 8 wherein the algorithm further comprises a temperature differential equation:
ΔT=T_{BA2}−T_{BA1}.

10. A method for normalizing body temperature of a subject comprising
a) providing raw body temperature (T_{R}) of a subject,
b) quantitatively normalizing the raw body temperature (T_{R}) with a temperature-normalizing algorithm wherein the algorithm comprises an equation containing at least one body temperature-affecting variable to obtain a normalized body temperature (T_{BA}).

11. The method of claim 10 wherein providing raw body temperature is via measurement of raw body temperature.

12. The method of claim 10 wherein the equation is
T_{BA}=T_{R}−0.104G+0.0107A+0.549(sin(2πt))−0.614(cos(2πt))−1.172,
wherein
T_{BA}=normalized body temperature,
T_{R}=raw body temperature,
G=gender (1=male, 2=female),
A=age (years), and
t=time of day (in decimal proportion of the day).

13. A method for determining a physiologically significant change in body temperature of a subject comprising
a) providing raw body temperature (T_{R}) of a subject;
b) providing data for a temperature-normalizing algorithm;
c) quantitatively normalizing the raw body temperature (T_{R}) with the algorithm wherein the algorithm comprises an equation containing at least one body temperature-affecting variable to obtain a normalized body temperature (T_{BA}); and
d) comparing the normalized body temperature (T_{BA}) to a second temperature.

14. The method of claim 13 wherein providing raw body temperature is via measurement of raw body temperature.

15. The method of claim 13 wherein the subject is a human or an animal.

16. The method of claim 13 wherein the physiological significant change in body temperature correlates to an immune response.

17. The method of claim 13 wherein the physiological significant change in body temperature correlates to an inflammatory disease.

18. The method of claim 13 wherein the physiological significant change in body temperature correlates to depression.

19. The method of claim 13 wherein the physiological significant change in body temperature correlates to ovulation.

20. The method of claim 13 wherein the equation is
T_{BA}=T_{R}−0.104G+0.0107A+0.549(sin(2πt))−0.614(cos(2πt))−1.172,
wherein
T_{BA}=normalized body temperature,
T_{R}=raw body temperature,
G=gender (1=male, 2=female),
A=age (years), and
t=time of day (in decimal proportion of the day).

21. The method of claim 19 wherein the providing data comprises provisions of numerical figures for gender, age, and time of day.

22. The method of claim 13 wherein the second temperature is a normalized body temperature from a different subject, a normalized body temperature of the same subject from a different time, or a baseline average population body temperature.

23. The method of claim 13 further comprising determining an algorithm for normalizing body temperature.

24. The method of claim 13 further comprising diagnosing or identifying a physiological condition or event based on the comparison of the normalized body temperature to the second temperature and on known correlations of body temperature and the physiological condition or event.

25. A method for determining febrile conditions in a subject comprising
a) taking or referring to a patient history including questions related to symptoms of fever;
b) performing a physical exam including at least measuring the patient's body temperature;
c) normalizing the patient's temperature measurement using a quantitative temperature-normalizing algorithm comprising an equation containing at least one body temperature-affecting variable; and
d) analyzing the history, physical exam, and normalized temperature information to determine the probability of fever based on known correlations of that information and fever.

26. A method for determining ovulation in a subject comprising
a) determining raw basal body temperature of a female subject;
b) normalizing the raw basal body temperature using a quantitative temperature-normalizing algorithm comprising an equation containing at least one body temperature-affecting variable;
c) charting the normalized basal body temperature over a period of time, and
d) identifying a rise in normalized basal body temperature which correlates with ovulation having occurred.

27. A method for diagnosing depression in subject comprising
a) taking or referring to a patient history including questions related to depression;
b) performing a physical exam including at least measuring the patient's body temperature;
c) normalizing the patient's temperature measurement using a quantitative temperature-normalizing algorithm comprising an equation containing at least one body temperature-affecting variable; and
d) analyzing the history, physical exam, and normalized body temperature information to determine the probability of depression based on known correlations of that information and depression.

28. A computer program product for normalizing body temperature, the program being embodied on a computer-readable medium, on which is carried the program comprising:
a code segment comprising a quantitative temperature-normalizing algorithm comprising an equation containing at least one body temperature-affecting variable.

29. The product of claim 28 wherein the algorithm comprises an equation
T_{BA}=T_{R}−0.104G+0.0107A+0.549(sin(2πt))−0.614 (cos(2πt))−1.172,
wherein
T_{BA}=normalized body temperature,
T_{R}=raw body temperature,
G=gender (1=male, 2=female),
A=age (years), and
t=time of day (in decimal proportion of the day).
